(19) United States
(12) Patent Application Publication
(10) Pub. No.: US 2010/0137829 A1
(43) Pub. Date: Jun. 3, 2010

(54) Injection Gateway

(76) Inventors: Henrik Bøje Nielsen, Lejre (DK); Jens Egebjerg Nielsen, Ringsted (DK); Orla Mathiasen, Soro (DK); Julie Grundtvig Theander, Olstykke (DK)

Correspondence Address: BRINKS HOFER GILSON & LIONE, P.O. BOX 10395, CHICAGO, IL, 60610, US

(21) Appl. No.: 12/525,191

(22) PCT Filed: Feb. 1, 2008
(86) PCT No.: PCT/EP08/51278

§ 371 Date: Jan. 15, 2010

Related U.S. Application Data
(60) Provisional application No. 60/899,075, filed on Feb. 2, 2007.
(60) Provisional application No. 60/899,062, filed on Feb. 2, 2007.
(60) Provisional application No. 60/899,143, filed on Feb. 2, 2007.

(30) Foreign Application Priority Data
Feb. 2, 2007 (PA) 2007-00179
Feb. 2, 2007 (PA) 2007-00185
Feb. 2, 2007 (PA) 2007-00191

(51) Int. Cl. A61M 5/32

(52) U.S. Cl. 604/506; 604/164.01; 604/164.08

(57) ABSTRACT

The current invention relates to an injection gateway (1) comprising a base part (2) which during use is secured to the skin (3) of a patient, a cannula (7) which is connected to the base part and which is inserted into the body of the patient during use, a gate (13) through which fluid is injectable into the gateway via a needle based injection device, and a fluid passageway (12) where fluid injected through the gate is communicated to the cannula. The injection gateway further comprises a closing mechanism (5), said closing mechanism having a first position where a needle inserted into the gateway can be inserted into the cannula and a second position where a needle inserted into the gateway is prevented from coming into contact with the cannula. In this way, the gateway allows the use of typical injection needles without the risk of piercing the cannula or penetrating too deeply into the body of the user.

The current invention relates to an injection gateway comprising a base part which during use is secured to the skin of a patient, a cannula which is connected to the base part and which is inserted into the body of the patient during use, a gate through which fluid is injectable into the gateway via a needle based injection device, said gate being located at a fixed position with regards to said base part, and a fluid passageway where fluid injected through the gate is communicated to the cannula.

An injection gateway is a device which is secured to the user for a certain period of time, for example three days. The user can then inject medications into his or her body via the injection gateway. The gateway therefore replaces repeated injections by syringes or injection pens. This reduces trauma to the patient's skin while simultaneously keeping the injection site free of infections. It is also much easier for people with fear of needles to use an injection gateway than to inject themselves directly.

### DESCRIPTION OF RELATED ART

Injection gateways are already known in many different types. In certain injection gateways, once the gateway is secured to the skin of the user, fluid is delivered to the gateway via an adapter attached to the gateway, see for example U.S. Pat. No. 4,755,173, WO 2004/101071, WO 02/053220 and U.S. 2004/0158207. However these devices require the use of a special adapter and are usually connected to an infusion pump via tubing. They are not suitable for use together with a needle based injection device such as an ordinary syringe. In addition, in many cases, in order to work with the tubing of the infusion pump, the mechanisms of the injection gateways becomes rather complex with complicated fluid paths inside the device which increase the risk of leaks in the device and/or increase the cost of the device due to the complicated seals which need to be integrated into the device.

Other types of gateways are designed to be used together with a special pen-type injector, see for example applicant's own disclosure WO 2006/097111. In the most common form, the gateway is arranged such that a needle on the pen-type injector pierces a septum on the gateway such that fluid can be injected through the septum and into the body of the user via the cannula which is inserted into the body of the user. However, devices of this kind require the use of a special pen-type injector device since the needle of the injection device should not come into contact with the cannula. If the needle comes into contact with the cannula, there is a risk that the needle could pierce the side of the cannula or scrape small plastic parts off the cannula which could end up in the body of the user. Furthermore, the use of short needles is preferred such that the risk that a user accidentally penetrates too deeply into his or her skin is minimized. Should a user use an ordinary syringe with the known gateways, the user will expose him or herself to the above mentioned risks.

### SUMMARY OF THE INVENTION

A first aspect of the present invention is to provide a gateway which is easy for the patient to place and to use for self-administration of drugs or other medicaments.

A second aspect of the present invention is to provide a gateway where the gateway after placement onto the patient's skin is noticed as little as possible by the patient when the patient is not actually injecting medication.

A third aspect of the present invention is to provide a gateway which allows the user to safely use an ordinary injection syringe without the risks ordinarily associated herewith.

A fourth aspect of the present invention to provide a gateway which allows the user to safely use an ordinary injection syringe while preventing the user from injecting the needle too deeply into his or her body.

A fifth aspect of the present invention is to provide a gateway which prevents the user from damaging the cannula with a needle inserted into the gateway after the gateway is secured to the user's skin.

A sixth aspect of the present invention is to provide a gateway which is simple and of low cost.

The injection gateway according to the current invention as claimed meets these aspects by providing an injection gateway as mentioned in the introductory paragraph which further comprises a closing mechanism having a first position where a needle is insertable into the gateway and insertable into the cannula and a second position where a needle of a needle based injection device is insertable into the gateway in order to inject fluid into the patient via the cannula but where said needle is prevented from coming into contact with the cannula. By providing such a closing mechanism, the user is prevented from sticking a needle into the cannula or sticking a needle too far into his or her body. The gateway according to this invention therefore reduces the risks normally associated with the use of an ordinary needle based injection device.

In one embodiment of such an injection gateway, the gateway can be arranged such that in the first position of the closing mechanism, a needle inserted into the gateway and into the cannula passes through the gate. The gateway could also be arranged such that in the second position of the closing mechanism, a needle inserted through the gate is prevented from coming into contact with the cannula. In this way, the access point for the different needles is the gate, thereby providing an effective seal between the environment outside the gateway and the user's body on the inside of the gateway. In a typical embodiment, the gate could comprise a pierceable septum.

In one embodiment, the fluid passageway can be arranged such that it establishes fluid communication between two chambers. The two chambers could be arranged such that the axis of the cannula passes through a first of the two chambers and not through a second of the two chambers. The chambers could furthermore be arranged such that needles inserted into the gateway when the closing mechanism is in its first position are inserted into the first chamber and that needles inserted into the gateway when the closing mechanism is in its second position are inserted into the second chamber. In this way, needles inserted into the gateway come into contact with two unique chambers.

In another embodiment, the closing mechanism could comprise a cover element having an access port through which a needle can be inserted into the gateway and which is displaceably connected to the base part.

The cover element could furthermore be arranged such that in the first position of the closing mechanism its access port is aligned with the axis of the cannula such that an inserter needle can be inserted through the access port and into the cannula. This position is useful during insertion of the cannula into the user's skin via an inserter needle which is arranged inside the cannula.

The cover element could also be arranged such that in the second position of the closing mechanism, the access port is not aligned with the axis of the cannula. In this way, a needle inserted into the gateway through the access port is prevented from coming into contact with the cannula.

The cover element could be rotatably connected to the base part about an axis. In this case, the access port in the cover element and the cannula could be arranged offset from the axis of rotation of the cover element. In this way, by rotating the cover element, the access port can be moved from a position where it is positioned aligned with the axis of the cannula, to a position where it is not aligned with the axis of the cannula.

The cover element could alternatively or simultaneously be connected to the base part in such a manner that it is displaceable along a path.

In certain embodiments, the closing mechanism could comprise a locking mechanism whereby the closing mechanism can be locked in the second position. This ensures that the gateway will only be used once. That is to say, the gateway will not be able to be removed from the user's body and then reapplied at a later point in time. The user will be required to use a new gateway.

In one embodiment, the locking mechanism could comprise a ratchet mechanism. In another embodiment, the locking mechanism could comprise an elastically biased protrusion element which engages with a corresponding recess when the closing mechanism is put into the second position of the closing mechanism. In one embodiment, the protrusion element is arranged on the cover element and the recess is arranged on the base part. The opposite case could also be imagined. Such mechanisms are simple and robust.

In one embodiment, the cover element could be arranged in such a way that it is aligned with the base element in the second position of the closing mechanism and not aligned with the base element in the first position of the closing mechanism. In this way, the user is given a clear visual signal when the device is “safe” and when it is “unsafe”. This prevents the user from using the device in its “unsafe” mode.

The closing mechanism could also comprise a blocking element which in the first position of the closing mechanism is displaced away from the axis of the cannula and in the second position of the closing mechanism is displaced over the axis of the cannula in order to block the entrance to the cannula.

The blocking element could be elastically biased towards a position where it covers the cannula opening. In such an embodiment, the blocking element could be prevented from covering the cannula opening in the first position of the closing mechanism in that it is held back by the inserter needle. The blocking element could however be biased in other ways than elastically, for example via gravity.

In one embodiment, the blocking element could be a ball which is released when the inserter needle is removed and which rolls into the opening of the cannula in order to block the entrance to the cannula.

In another embodiment, the blocking element could be displaced due to an actuation element which responds to warmth and/or moisture when the gateway is removed from its packaging and secured to the user. This provides a closing mechanism which is to a certain extent “automatic” and ensures that the gateway is put into its safe mode without interference by the user.

It could furthermore be imagined that a gateway as described above could be part of a set which comprises an injection gateway and an inserter needle already arranged in the cannula of the gateway. The set could further comprise an inserter device for allowing a user to more easily insert the cannula into his or her body.

Such a set could also comprise a lid which covers the gateway during the times when the gateway is secured to the user but not being actively used as an injection gateway.

A gateway of the kind described above could be used according to a method which comprises the steps of placing an inserter needle through a cannula of the injection gateway, inserting the inserter needle and cannula beneath the skin of the user, securing the injection gateway to the user, removing the inserter needle, displacing a closing mechanism such that access to the cannula with a needle is prevented, and injecting fluid from a needle based injection device into the user via the injection gateway without further displacement of the closing mechanism.

In one such method, the step of displacing the closing mechanism could occur automatically after removal of the inserter needle from the injection gateway.

In another embodiment of such a method, the step of displacing the closing mechanism could comprise the step of the user rotating a cover element with respect to a base element of the gateway.

It should be emphasized that the term “comprises/comprising” when used in this specification is taken to specify the presence of stated features, integers, steps or components but does not preclude the presence or addition of one or more other features, integers, steps, components or groups thereof. For example, the description describes a closing mechanism having two positions. However, it should be obvious to the person skilled in the art that the closing mechanism could have more than two positions even though only two positions are explicitly mentioned in the claims and the description.

### BRIEF DESCRIPTION OF THE DRAWINGS

In the following, the invention will be described in greater detail with reference to embodiments shown by the enclosed figures. It should be emphasized that the embodiments shown are used for example purposes only and should not be used to limit the scope of the invention.

FIG. 1 shows a cross section view of a first embodiment of an injection gateway mounted in an injector device comprising an injector needle.

FIG. 2 shows a cross section view of the first embodiment of an injection gateway secured to the skin of a user and where the closing mechanism is in its first position.

FIG. 3 shows a cross section view of the first embodiment of an injection gateway secured to the skin of a user and where the closing mechanism is in its second position.

FIG. 4 shows a perspective view of the first embodiment of an injection gateway where the closing mechanism is in its first position.

FIG. 5 shows a perspective view of the first embodiment of an injection gateway where the closing mechanism is in its second position.

FIG. 6 shows a perspective cross section view of the first embodiment of an injection gateway where the closing mechanism is in its first position. Note the septum has been removed from FIG. 6 in order to make the inner details easier to see.

FIG. 7 shows a perspective cross section view of the first embodiment of an injection gateway where the closing mechanism is in its second position. Note the septum has been removed from FIG. 7 in order to make the inner details easier to see.

FIG. 8 shows a schematic cross section view of a second embodiment of an injection gateway where the closing mechanism is in its first position.

FIG. 9 shows a schematic cross section view of the second embodiment of an injection gateway where the closing mechanism is in its second position.

FIG. 10 shows a schematic cross section view of a third embodiment of an injection gateway where the closing mechanism is in its first position.

FIG. 11 shows a schematic cross section view of the third embodiment of an injection gateway where the closing mechanism is in its second position.

FIG. 12 shows a schematic perspective cross section view of a fourth embodiment of an injection gateway where the closing mechanism is in its first position.

FIG. 13 shows a schematic perspective cross section view of the fourth embodiment of an injection gateway where the closing mechanism is in its second position.

FIGS. 14a-14d show another approach to minimizing the risks associated with the use of an ordinary needle based injection device in commonly available injection gateways.

### DETAILED DESCRIPTION OF THE EMBODIMENTS

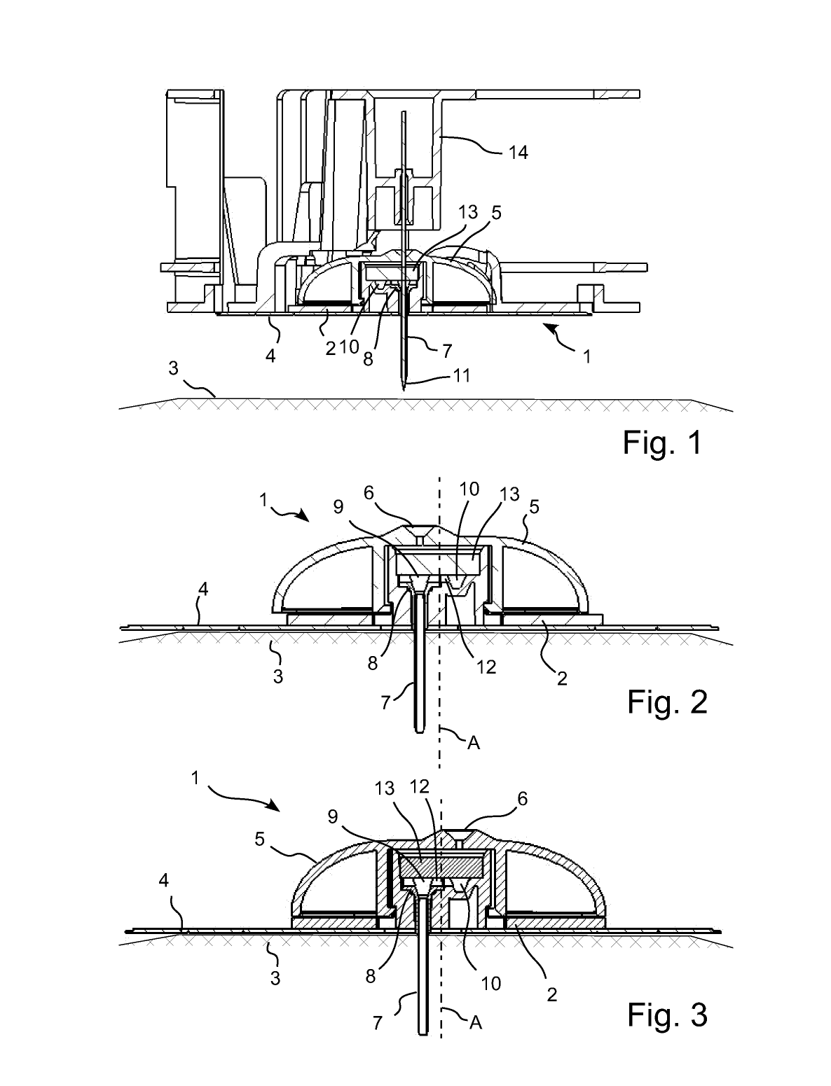

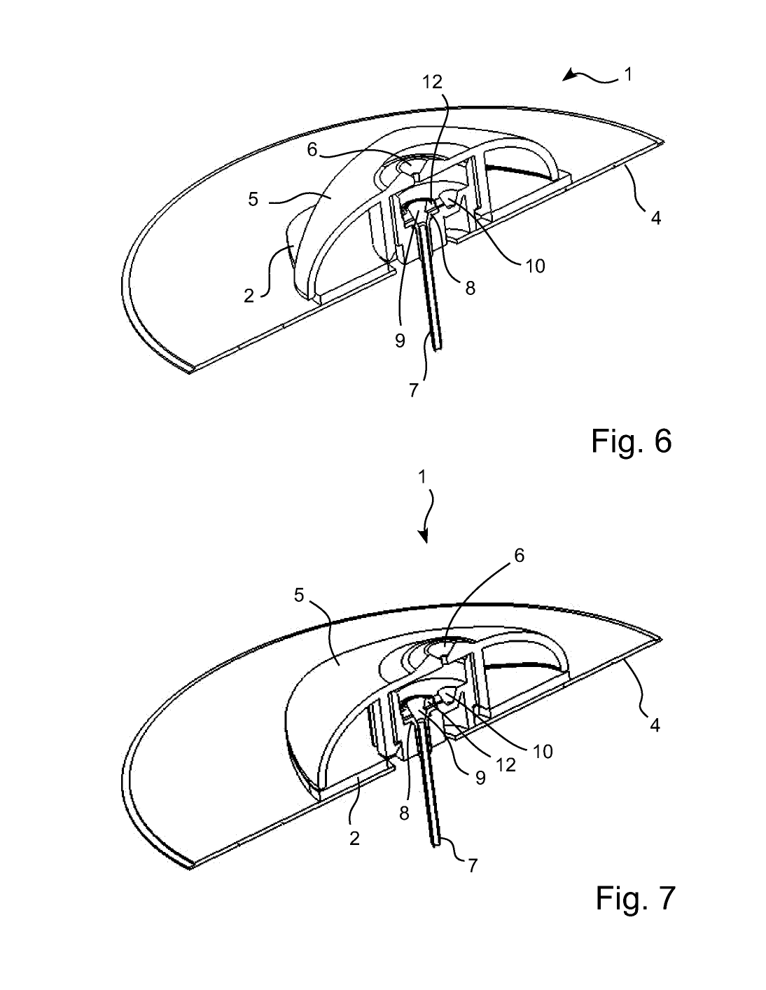

FIGS. 1-7 show some different views of a first embodiment 1 of an injection gateway according to the invention as claimed. The gateway 1 comprises a base part 2 which is secured to the user's skin 3 via an adhesive layer 4 attached to the bottom of the base part 2.

A cover element 5 is rotatably connected to the base part 2 about an axis A. The cover element 5 has an access port 6 through which a needle can be inserted in order for the needle to gain access to the interior of the gateway. The access port 6 is arranged offset from the axis A. In this way, when the cover element is rotated about the axis, the access port 6 is both rotated and moved along a circular path.

The base element 2 comprises a cannula 7 which is inserted into the user's body when the gateway is in use. The cannula is therefore in fluid communication with the interior of the user's body during use. In the current embodiment the cannula is made of a soft plastic material as is well known to the person skilled in the art. The soft plastic cannula can be left in the user's body without causing discomfort for the user.

The cannula 7 is connected to the base part 2 via a bushing 8. The bushing is typically made of a plastic material which fuses the base part and the cannula together. However, in certain cases, it could be imaged that the bushing was made of a metal material. This will prevent damage to the gateway by the needle. In other cases, the cannula can be fused directly to the base part, thereby eliminating the need for the bushing.

The base part further comprises two chambers 9, 10. The first chamber 9 is in direct fluid communication with the cannula 7 and is arranged such that the axis of the cannula passes through the first chamber 9. This allows an inserter needle 11 which needs to be inserted through the cannula during the insertion of the cannula into the body of the user to pass through the first chamber. The second chamber 10 is arranged offset from the axis of the cannula. The second chamber 10 is however in fluid communication with the cannula via a fluid passageway 12. In this way, fluid inserted into the second chamber reaches the cannula and therefore also reaches the body of the user. It should be noted that in the current embodiment, the fluid passageway is established between the first chamber and the second chamber, however, it could be imaged that the second chamber was also connected directly to the cannula.

The base part 2 also comprises a gate 13 in the form of a pierceable septum. The function of the gate 13 is to allow a needle to penetrate the septum in order to inject fluid into the gateway, but to prevent bacteria and other unwanted matter from getting into the inside of the gateway. The use of a gate such as a septum for this purpose is well known to the person skilled in the art and will not be further discussed here.

As can be seen from the figures, the two chambers 9,10 are also arranged offset from the axis of rotation of the cover element. In the current embodiment the two chambers are arranged 180 degrees apart with respect to the axis of rotation of the cover element. In addition, the two chambers are arranged to be offset the same amount from the rotation axis A as the access port on the cover element. In this way, when the cover element is rotated about the axis A the access port 6 can be made to either give a needle access to the first chamber 9 (FIGS. 1, 2, 4 and 6) or to the second chamber 10 (FIGS. 3, 5 and 7). In this way, the cover element acts as a type of closing mechanism having at least two positions. In one of the positions (first position) the access port is lined up with the first chamber and in another position (second position) the access port is lined up with the second chamber.

When the cover element is in the first position, a needle can be inserted through the access port, through the septum, through the first chamber and through the cannula. This position is used during the insertion of the device. It could be imagined that the gateway is delivered to the user with the cover element in the first position and with an inserter needle already inserted through the cannula. In this way, the user does not risk damaging the cannula by inserting the inserter needle into the gateway by him or herself.

When the gateway has been secured to the user and the cannula inserted into the user's body, then the inserted needle can be removed and the cover element rotated into the second position. In the second position, a needle can be inserted through the access port in the cover, through the septum and into the second chamber 10. However, the needle is prevented from reaching the cannula. The user can therefore use an ordinary needle based injection device without the risks which are typically associated with this.

FIG. 1 shows the gateway 1 attached to an “inserter device” 14. Inserter devices are well known to the person skilled in the art and can take many forms. The inserter device 14 is typically used in that the gateway is loaded into the inserter device, the device is primed and placed over the insertion location on the body of the user. The device is then released and the gateway is inserted into the body of the user. This allows for a simple insertion process which is relatively comfortable for the user. Once the gateway has been secured to the body of the user, the inserter device along with the inserter needle can be removed.

It should also be mentioned that the cover element is provided with an elastically biased hook which snaps into a groove in the base element when the cover element is rotated to the second position. In this way, the hook locks the cover element preventing further rotation. This ensures that the device can only be used once with an inserter needle. After the cover element has been rotated into the second position, then the cover element is locked and needles inserted into the device will not come into contact with the cannula.

It should also be mentioned that the first embodiment comprises an external cover element which is arranged on the outside of the base part. However, in another embodiment (not shown), the cover element could be arranged inside the base part. In this case, the cover element could be displaced via an actuation element which is accessible to the user through an opening in the base part. For example, the cover element could be attached to a protrusion which is arranged in a slot in the base part. The user could therefore displace the cover element by displacing the protrusion in the slot.

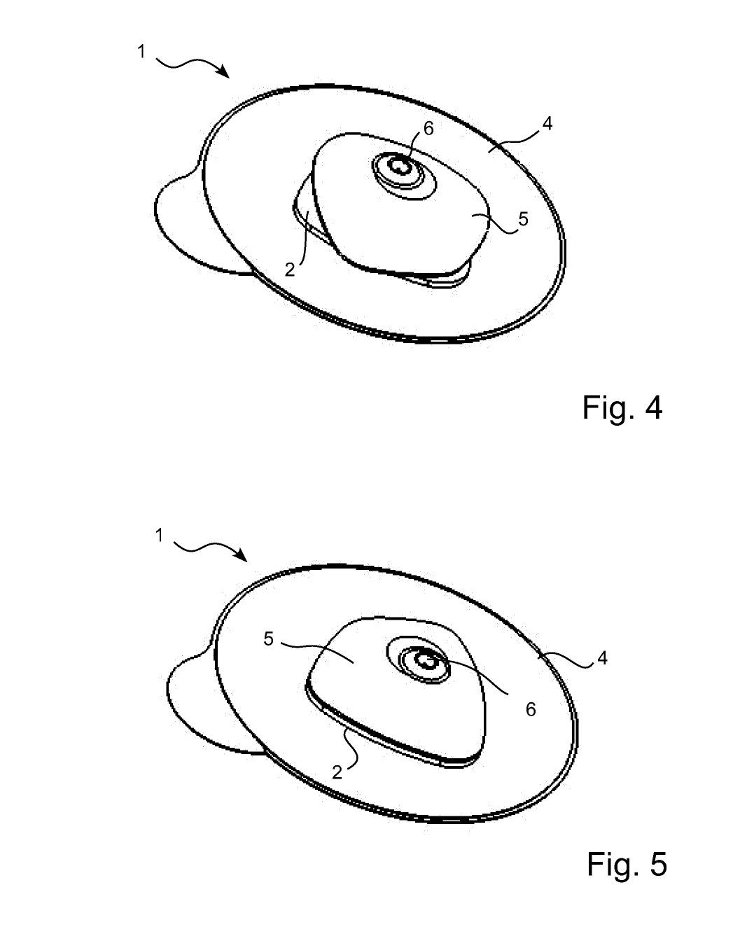

FIGS. 4 and 5 show the gateway in perspective from the top side of the device. As can be seen, the shape of the cover element 5 is not round, but looks a bit like a triangle. The base part is formed with the same outline. In this way, when the cover element is rotated, there is one position where the outline of the cover element and the outline of the base element match. In the other positions of the cover element, the user can see that the cover element and the base element are not aligned. This gives a visual signal to the user when the device is in the “safe position”. It should be obvious to the person skilled in the art that many other shapes would also provide this feature.

It can also be mentioned with respect to the first embodiment 1 that the cover element is arranged rotatable about an axis, however, the cover element could also be arranged to be displaceable along a path. For example, the user could slide the cover a short distance between the first position and the second position. Furthermore, a combination of rotation and displacement could also be possible.

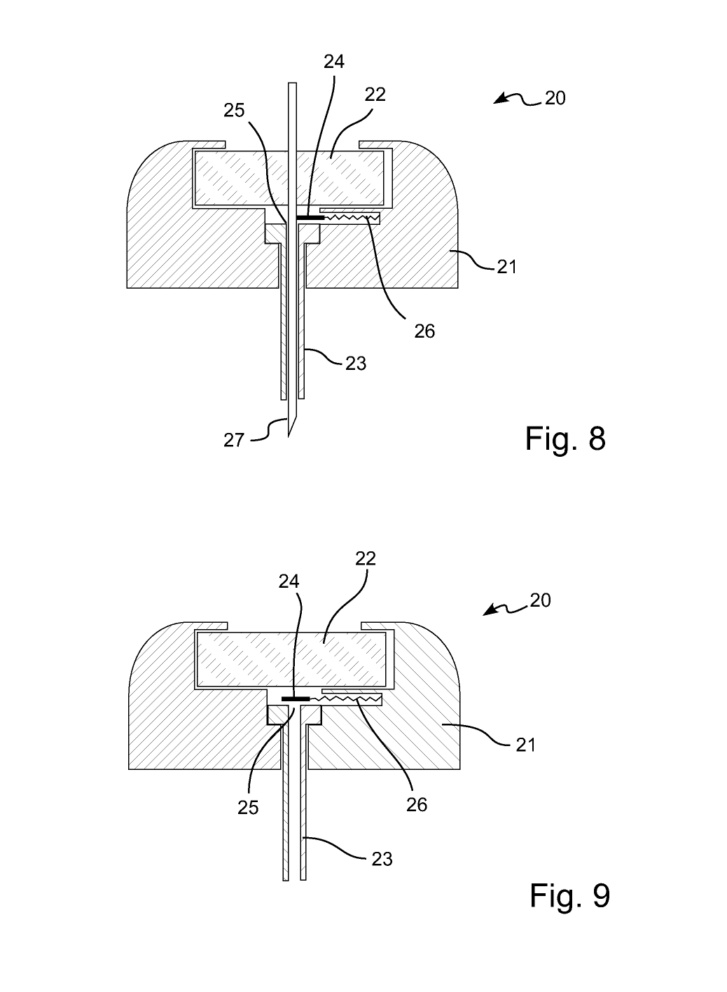

FIGS. 8 and 9 show a second embodiment 20 of a gateway according to the invention. In this embodiment, the gateway comprises a base element 21, a septum 22, and a cannula 23 attached to the base part. The gateway furthermore comprises a blocking element 24 biased towards the cannula opening 25 by a spring 26. The gateway is delivered to the user with an inserter needle 27 arranged in the cannula 23. The blocking element 24 is pressed against the inserter needle 27 by the spring 26. When the inserter needle is removed after securing the gateway to the user's skin, the blocking element 24 springs forward and blocks the entrance to the cannula. In this way, a needle inserted through the septum after removal of the inserter needle, will not be able to gain access to the cannula. However, fluid inserted into the gateway via the injection needle will be able to flow around the blocking element and into the cannula. FIG. 8 shows the gateway as it is delivered to the user and FIG. 9 shows the gateway after removal of the inserter needle.

It should be obvious to the person skilled in the art that the blocking element and the spring can be formed in many different ways. The spring element could for example be a plastic beam which is bent away from the cannula opening by the inserter needle. The person skilled in the art of mechanical components should be able to develop many different blocking elements which provide the same benefits as the embodiment shown in the figures.

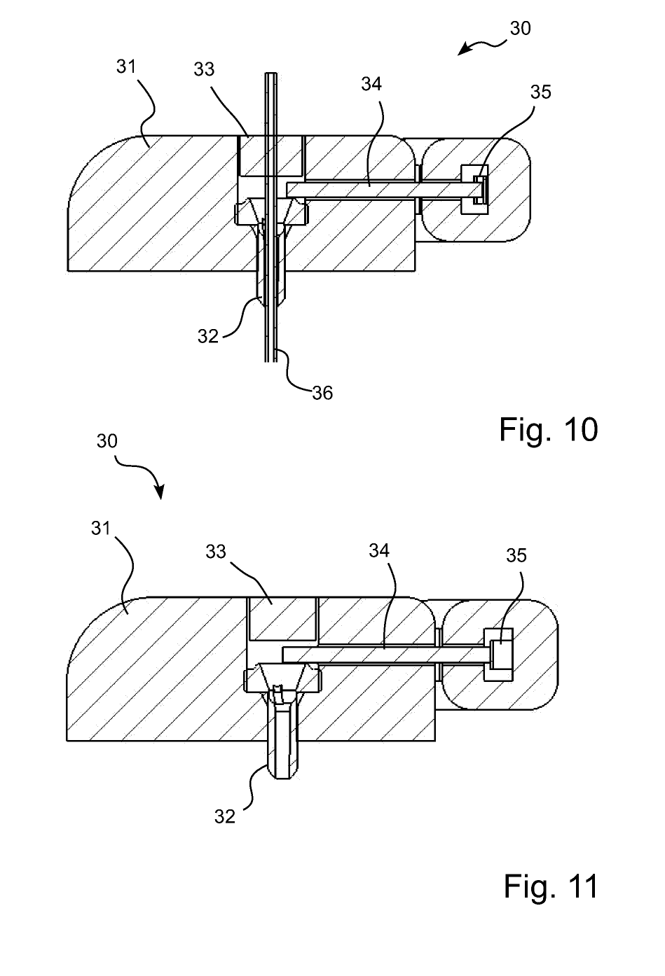

FIGS. 10 and 11 show a third embodiment 30 of a gateway according to the invention. This embodiment 30 also comprises a base element 31, a cannula 32 attached to the base element, a septum 33 and a blocking element 34. The blocking element is fastened to the base element via an actuating element 35. The actuating element is formed from a material which expands when exposed to moisture and warmth. When the gateway is packed in its sterile packaging, it is protected from moisture and can be kept cool. As soon as the package is opened and the gateway secured to the user, the material is exposed to moisture and warmth and begins to expand, thereby causing the blocking element to move forward and cover the opening to the cannula.

FIG. 10 shows the inserter needle 36 inserted in the device. FIG. 11 shows the gateway when secured to the skin of the user.

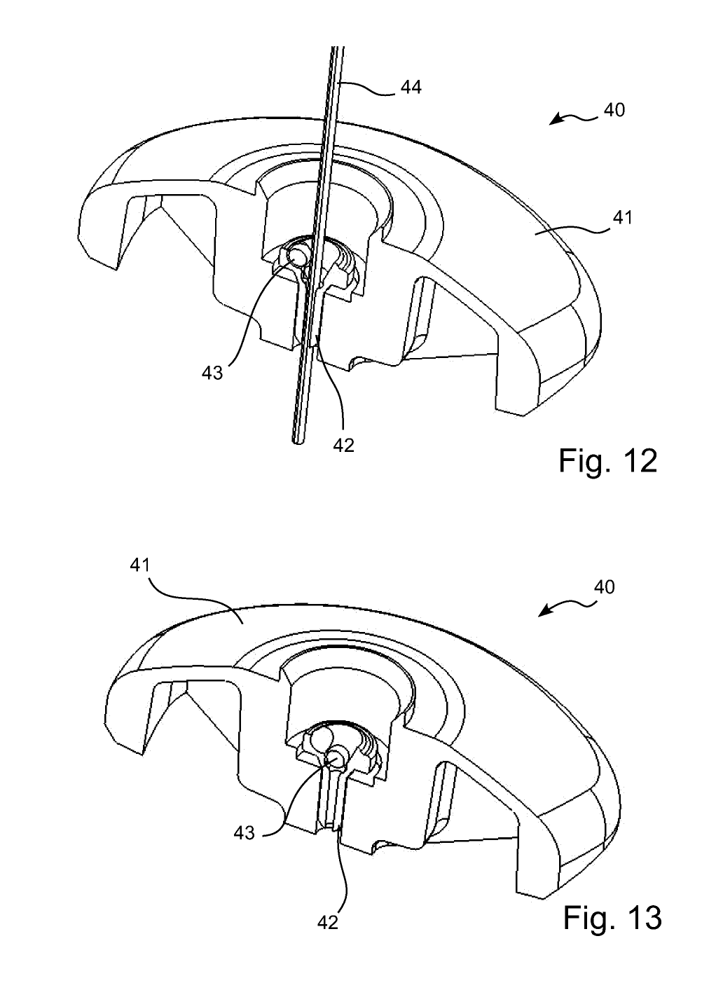

FIGS. 12 and 13 show a fourth embodiment 40 of a gateway according to the invention. In this embodiment, the gateway also comprises a base element 41, a cannula 42, a septum (not shown) and a blocking element 43. In this case, the blocking element is a small ball 43 which is held in place by the inserter needle 44. When the inserter needle is removed, the ball rolls into the opening of the cannula, thereby blocking access to the opening of the cannula. It should be noted that fluid is permitted to pass the ball in the second position of the ball.

It should be noted that in the above described embodiments, a single septum was used as a gate. In other words, both the inserter needle and the injection needles were inserted through the same septum. However, the gate could comprise multiple separate areas. For example one could imagine two separate septums, one for the inserter needle port and one for the injection needle port.

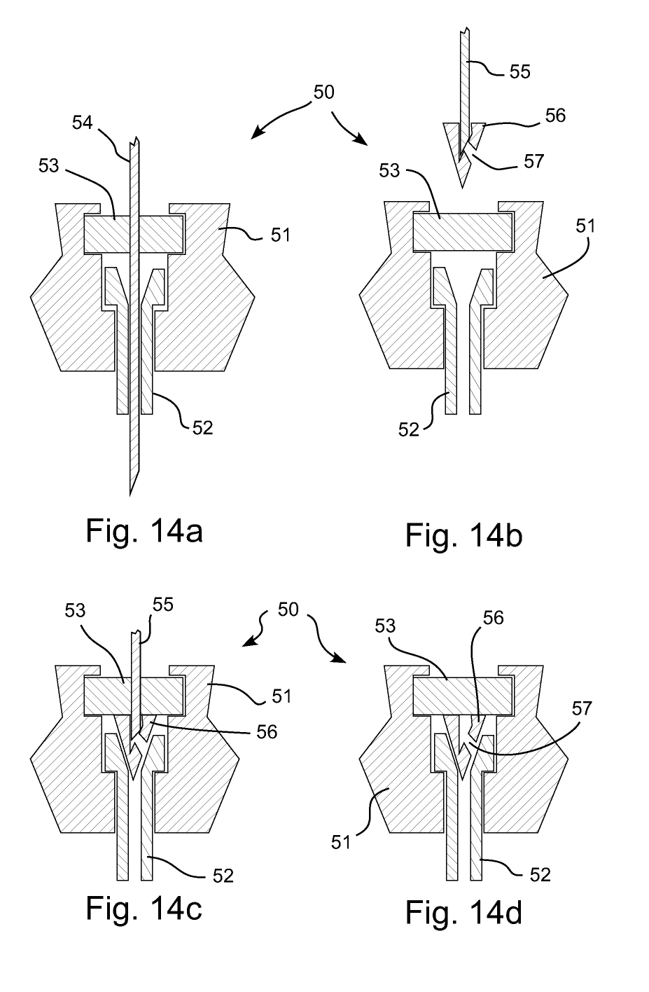

FIGS. 14a-14d show an embodiment of a system which can also be used to minimize the risks associated with the use of needle based injection devices. The figures show an injection gateway 50 comprising a base part 51, a cannula 52 and a septum 53. FIG. 14a shows an inserter needle 54 inserted in the gateway. The gateway 50 as shown in FIG. 14a is ready to be attached to a user. Once the gateway is secured to the user, the inserter needle is removed.

FIG. 14b shows a needle 55 of a typical needle based injection device. Before use, the user attaches a wedge shaped blocking element 56 to the tip of the needle 55. The user then inserts the needle 55 and wedge shaped blocking element 56 through the septum as shown in FIG. 14c. Due to the wedge shaped blocking element 56, the needle 55 is prevented from damaging the cannula and prevented from penetrating too deep in the user. When the user removes the needle 55 from the septum, the wedge shaped blocking element 56 remains in the gateway. This is shown in FIG. 14d. The next time that the user uses the gateway, the blocking device 56 is still in the device ready to block the needle. It should be noted that fluid can flow around the blocking element or as shown in the figures, a passageway 57 could be arranged in the blocking element 56.

In the above described embodiments, the cannula has been a soft plastic cannula. However, in certain cases, it could be imagined that the cannula is a thin needle made of metal. This reduces the risk of damage to the cannula from injection needles, but might be a bit less comfortable to the user. The metal cannula could however be inserted into the body of the user without the need for an inserter needle.

It is to be noted that the figures and the above description have shown the example embodiments in a simple and schematic manner. The internal details have not been shown in great detail since the person skilled in the art should be familiar with these details and they would just unnecessarily complicate this description.

It should also be mentioned that the current specification describes a number of independent inventions which could form parts of divisional applications. For example, the metal bushing arranged between the cannula and the base part could be used in many other gateways in order to prevent damage to the cannula from the injection needle. Another example is the cannula made from metal. This embodiment could be used on other types of gateways than the ones claimed in the current invention.

In addition, the embodiments shown in this specification should not be used to limit the scope of the invention unnecessarily. There are other embodiments of injection gateways which fall in under the scope of the current claim set. For example, the embodiments described above all show a gateway where the inserter needle pierces a septum. However, it could be imagined that the gateway has an open port for the inserter needle which is closed and sealed via the closing mechanism in the second position of the closing mechanism. For example the cover element could be equipped with a seal and an opening. When the gateway is in its “insertion mode”, the opening in the cover is arranged over the opening to the cannula. When the gateway is put into its “user mode”, the cover element is displaced such that the opening is displaced over a septum used for injections from a normal needle based injection device and the seal on the cover is displaced over the cannula opening, thereby preventing access to the cannula. The seal furthermore prevents unwanted bacteria and/or other unwanted matter from reaching the inside of the gateway. The person skilled in the art should be able to define the scope of the current claim set using his knowledge and the current specification.

1. Injection gateway comprising:
a base part secured to the skin of a patient during use,
a cannula connected to the base part and inserted into the body of the patient during use,
a gate comprising a pierceable septum through which fluid is injectable into the gateway via a needle-based injection device, said gate located at a fixed position in relation to said base part,
a fluid passageway operably connecting the gate to the cannula, and
a closing mechanism, said closing mechanism having a first position where a needle is insertable into the gateway and insertable into the cannula and a second position where a needle of a needle based injection device is insertable into the gateway to inject fluid into the patient via the cannula but where said needle is prevented from contacting the cannula,
wherein the gate is located at the same position in relation to the base part when the closing mechanism is in the first position and when the closing mechanism is in the second position.

2. An injection gateway according to claim 1, wherein in the first position of the closing mechanism, a needle inserted into the gateway and into the cannula passes through the gate.

3. An injection gateway according to claim 1, wherein in the second position of the closing mechanism, a needle inserted through the gate is prevented from coming into contact with the cannula.

4. An injection gateway according to claim 1, wherein the fluid passageway establishes communication between two chambers.

5. An injection gateway according to claim 4, wherein the two chambers are arranged such that an axis of the cannula passes through a first of the two chambers and not through a second of the two chambers.

6. An injection gateway according to claim 4 wherein needles inserted into the gateway when the closing mechanism is in the first position are inserted into the first chamber and needles inserted into the gateway when the closing mechanism is in the second position are inserted into the second chamber.

7. An injection gateway according to any claim 1, wherein the closing mechanism comprises a cover element having an access port through which a needle can be inserted into the gateway and said cover element is displaceably connected to said base part.

8. An injection gateway according to claim 7, wherein said cover element is arranged such that in the first position of the closing mechanism said access port is aligned with the cannula such that an inserter needle can be inserted through the access port and into the cannula.

9. An injection gateway according to claim 7 wherein said cover element is arranged such that in the second position of the closing mechanism said access port is not aligned with the cannula, such that a needle inserted into the gateway through the access port is prevented from coming into contact with the cannula.

10. An injection gateway according to claim 7 wherein said cover element is rotatably connected to the base part about an axis.

11. An injection gateway according to claim 10, wherein the access port in the cover element and the cannula are arranged offset from the axis of rotation of the cover element.

12. An injection gateway according to claim 7, wherein said cover element is connected to the base part so that the cover element is displaceable along a path.

13. An injection gateway according to any claim 1, wherein the closing mechanism comprises a locking mechanism, the closing mechanism can be locked in the second position.

14. An injection gateway according to claim 13, characterized in that the locking mechanism comprises a ratchet mechanism.

15. An injection gateway according to claim 13, wherein the locking mechanism comprises an elastically biased protrusion element which engages with a corresponding recess when the closing mechanism is in the second position of the closing mechanism.

16. An injection gateway according to claim 7, wherein the cover element is aligned with the base element in the second position of the closing mechanism and not aligned with the base element in the first position of the closing mechanism.

17. An injection gateway according to claim 1, wherein said closing mechanism comprises a blocking element which in the first position of the closing mechanism is displaced away from an axis of the cannula and in the second position of the closing mechanism is displaced over an axis of the cannula to block an entrance to the cannula.

18. An injection gateway according to claim 17, wherein said blocking element is elastically biased towards a position where the blocking element covers the cannula opening.

19. An injection gateway according to claim 17 wherein the blocking element is prevented from covering the cannula entrance in the first position of the closing mechanism by the needle.

20. An injection gateway according to claim 17 wherein said blocking element is a ball which is released when the needle is removed and the ball rolls into the opening of the cannula in order to block the entrance to the cannula.

21. An injection gateway according to claim 17, wherein the blocking element is displaced due to an actuation element which responds to warmth or moisture when the gateway is removed from a packaging and secured to the user.

22. A set comprising an injection gateway according to claim 1 and an inserter needle arranged in the cannula of the gateway.

23. A set according to claim 22, where said set further comprises an inserter device.

24. A set according to claim 22, where said set further comprises a lid which covers the gateway during the times when the gateway is secured to the user but not being actively used as an injection gateway.

25. A method of using an injection gateway comprising the steps of placing an inserter needle through a cannula of the injection gateway, inserting the inserter needle and cannula beneath the skin of the user, securing the injection gateway to the user, removing the inserter needle, displacing a closing mechanism such that access to the cannula with a needle is prevented, and injecting fluid from a needle based injection device into the user via the injection gateway without further displacement of the closing mechanism.

26. A method of using an injection gateway according to claim 25, wherein the step of displacing the closing mechanism occurs automatically after removal of the inserter needle from the injection gateway.

27. A method of using an injection gateway according to claim 25, wherein the step of displacing the closing mechanism comprises rotating a cover element in relation to a base element of the gateway.
